(19) United States
(12) Patent Application Publication
(10) Pub. No.: US 2014/0330175 A1
(43) Pub. Date: Nov. 6, 2014

(54) SYSTEM AND METHOD FOR COUPLING AND DEPTH CONTROL FOR ULTRASOUND

(71) Applicant: SonaCare Medical, LLC, Charlotte, NC (US)

(72) Inventor: Mark Carol, Charlotte, NC (US)

(73) Assignee: SonaCare Medical, LLC, Charlotte, NC (US)

(21) Appl. No.: 14/269,441

(22) Filed: May 5, 2014

Related U.S. Application Data
(60) Provisional application No. 61/818,992, filed on May 3, 2013.

(51) Int. Cl. A61N 7/00

(52) U.S. Cl. CPC A61N 7/00; A61N 2007/0004
USPC 601/2

(57) ABSTRACT

An ultrasound system includes a sheath having a distal end and a proximal end. The distal end may include an opening configured to allow the passage of a fluid between the sheath and a chamber in a patient. The proximal end may include one or more ports configured to allow the passage of the fluid into and out of the sheath. A probe may include a shaft and an ultrasound transducer coupled to the shaft. The probe may be configured for insertion into the proximal end of the sheath.

### RELATED APPLICATION DATA

This application claims priority to U.S. Provisional Patent Application No. 61/818,992, filed May 3, 2013 and entitled “COUPLING AND DEPTH CONTROL FOR FOCUSED ULTRA SOUND DEVICES BY ADJUSTING CHAMBER SIZE,” which is hereby incorporated by reference in its entirety.

### BACKGROUND

Focused ultrasound (“FUS”) devices use ultrasound (“US”) transducers to deliver generally thermal or cavitational dose to a small well-defined small spot at some fixed or focal distance from the transducer surface. One or more ultrasound crystals are combined to form a transducer that can be geometrically or electronically focused at a point distant from the surface of the transducer thereby concentrating the US energy at the focal spot. This concentration of sound energy results in cavitational and thermal damage to the region of focus and can be used, among other things, to destroy cancerous tissue.

One way to deliver a thermal dose to a larger region is to move the transducer so that the small spot of thermal dose is scanned over the region that is to receive thermal or cavitational dose. Another way is to move the patient relative to the transducer. The latter approach is often used in extracorporeal devices where the transducer is located outside the patient. Such is the case with the EXABLATE™ system. The former approach is used often in devices where the transducer is located inside the patient. Such is the case with devices such as the SONATHERM™ and SONABLATE™ devices.

In devices where the transducer is introduced into the patient and is moved potentially relative to the patient, it is typically deployed in a probe. The probe will typically include a means for coupling the transducer to the tissue to be treated—coupling involves providing a continuous water path between the transducer and the tissue being treated. In addition, the coupling mechanism is used to control the depth of the focal point of the transducer in the region of interest; increasing the depth of the water contained by the means for coupling allows the focal point of the transducer to be moved deeper or shallower in the tissue to which it is coupled. The probe may contain an US transparent window through which the thermal US energy passes. This window typically is larger than the transducer. The transducer can be moved around inside the window in order to deliver dose to a region greater in width and or length than the size of the transducer itself. Rigid or flexible shaft-based drive systems can be employed to move the transducer inside the probe around in the window so that that the spot of thermal dose can be scanned over the region that is to receive thermal or cavitational dose without the need to move the probe itself around inside the patient. This reduces the amount of trauma to which the patient would be subjected and the loss of tissue coupling that would occur if the probe itself were moved around. However, in order to deliver dose in a precisely controlled manner, the transducer needs to be positioned correctly and rigidly relative to an acoustic window that will stay in contact with the target tissue and can be adjusted in volume to move the focal spot relative to the target tissue, requiring rigidity of the means for coupling that increases the diameter of the complete probe system.

Prior art systems and methods can undesirably limit the portals through which the probe can be introduced. For instance, one means of accessing bladder wall tumors is to pass instrumentation through the urethra, a relatively small diameter passage that limits the size of the instruments that can be utilized. The need to include a rigid sheath surrounding the probe that is secured to the probe may limit the ability to introduce a probe large enough to treat the target lesion.

### BRIEF DESCRIPTION OF THE DRAWINGS

The following detailed description of the invention will be better understood when read in conjunction with the appended drawings. For the purpose of illustrating the invention, there are shown in the drawings various illustrative embodiments. It should be understood, however, that the invention is not limited to the precise arrangements and instrumentalities shown. In the drawings:

FIG. 1A is a schematic diagram of an ultrasound device according to an exemplary embodiment of the present disclosure;

FIG. 1B is a schematic diagram of a probe of the ultrasound device shown in FIG. 1A;

FIG. 1C is a schematic diagram of a sheath of the ultrasound device shown in FIG. 1C;

FIG. 2 is a schematic diagram of an ultrasound device including a balloon according to an exemplary embodiment of the present disclosure;

FIG. 3 illustrates a flowchart for delivering an ultrasound treatment according to an exemplary embodiment;

FIGS. 4A-4B are schematic diagrams illustrating the operation of an ultrasound device in a natural chamber according to an exemplary embodiment of the present disclosure;

FIGS. 5A-5B are schematic diagrams illustrating the operation of an ultrasound device in an artificial chamber according to an exemplary embodiment of the present disclosure; and

FIG. 6 illustrates an exemplary computing environment that may be used with the method and device for ultrasound treatment according to the present disclosure.

### DETAILED DESCRIPTION

While methods, apparatuses, and devices are described herein by way of examples and embodiments, those skilled in the art will recognize that the methods, apparatuses, and devices for delivering an ultrasound treatment are not limited to the embodiments or drawings described. It should be understood that the drawings and description are not intended to be limited to the particular form disclosed. Rather, the intention is to cover all modifications, equivalents and alternatives falling within the spirit and scope of the appended claims. Any headings used herein are for organizational purposes only and are not meant to limit the scope of the description or the claims. As used herein, the word “may” is used in a permissive sense (i.e., meaning having the potential to) rather than the mandatory sense (i.e., meaning must). Similarly, the words “a,” “an” and “the” mean “at least one,” and the words “include,” “includes” and “including” mean “including, but not limited to.”

Various non-limiting embodiments of the device and method for delivering ultrasound treatment are described hereinafter with reference to the figures. It should be noted that the figures are not drawn to scale. It should also be noted that the figures are not intended to facilitate the description of specific embodiments of the disclosure. They are not intended as an exhaustive description of the disclosure or as a limitation on the scope of the disclosure. In addition an aspect described in conjunction with a particular embodiment of the present disclosure is not necessarily limited to that embodiment and can be practiced in any other embodiments of the present disclosure. It will be appreciated that while various non-limiting embodiments of the disclosure are described in connection with radiation treatment of tumors, the claimed invention has application in other industries and to targets other than cancers.

There is a need for a device, system, method and/or means of delivering US and/or FUS to target tissue found in natural or artificial chambers of a patient that does not require 1) a means of tissue coupling integral or secured to the probe itself, or 2) to target tissue where there is tissue surrounding the target tissue that will prevent movement of the probe once it is in place.

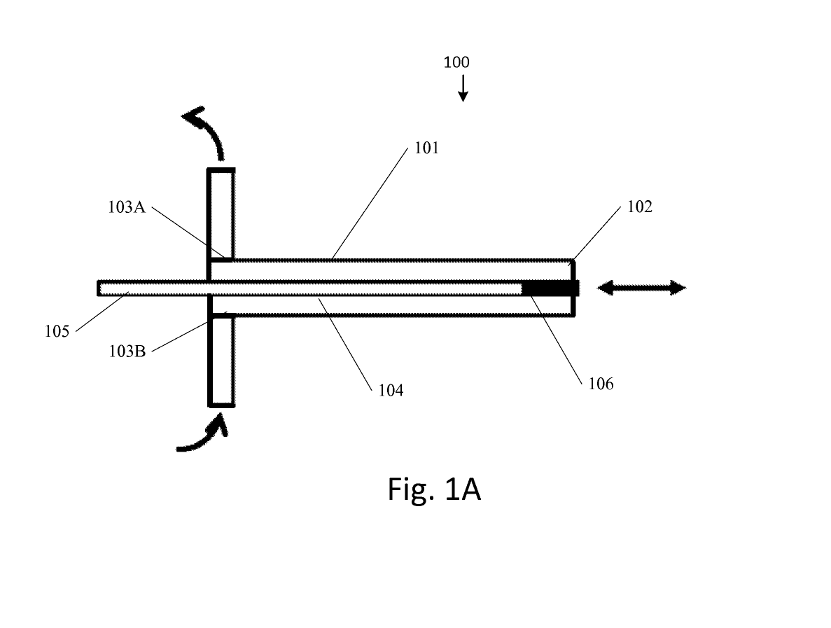

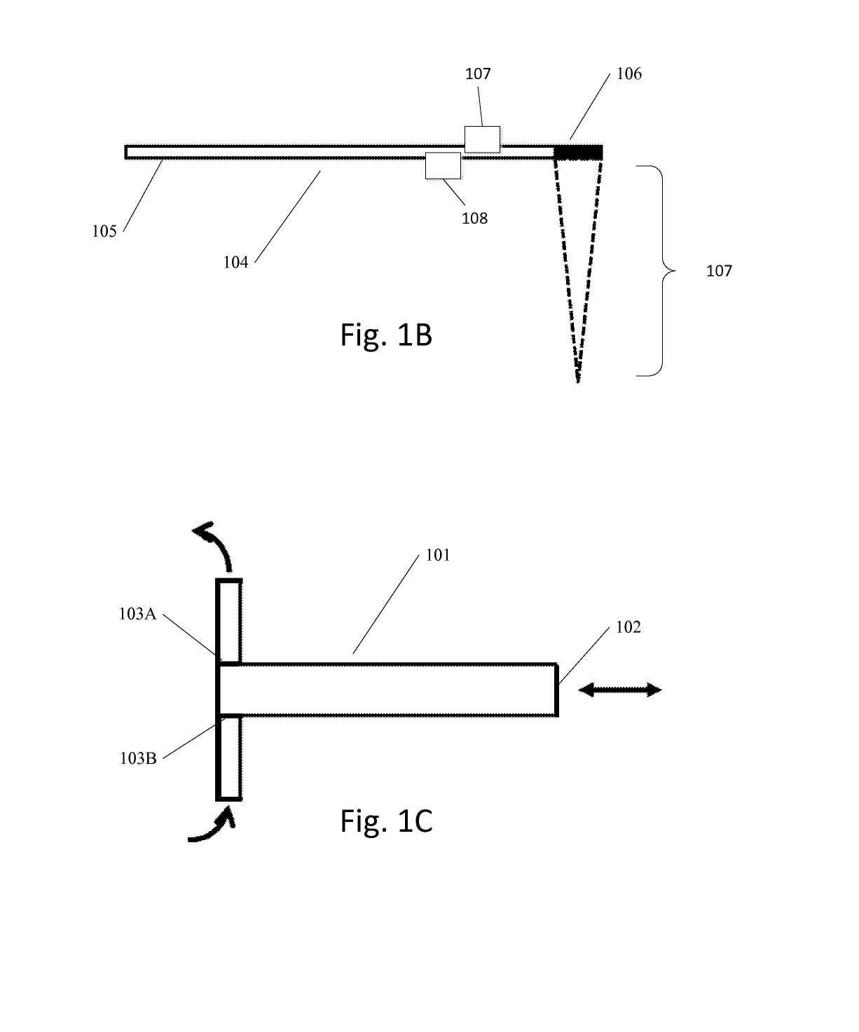

FIG. 1A illustrates a schematic diagram of an US and/or FUS device or system, generally designated 100, according to an exemplary embodiment of the present disclosure. The FUS device 100 may include a sheath 101 having a distal end, an opposing proximal end and a passageway therebetween. The distal end may include an opening 102 configured to allow the passage of at least some fluid between the sheath 101 and a chamber in a patient. The proximal end of the FUS device 100 may include one or more ports 103A, 103B configured to allow the passage of at least some of the fluid into and out of the sheath 101.

Referring to FIGS. 1A and 1B, the FUS device 100 may include a probe 104 comprising a drive shaft 105 and one or more transducers 106 coupled to the drive shaft 105. As shown in FIG. 1A, the probe 104 may be configured for insertion at least partially or completely into the proximal end of the sheath 101.

FIG. 1B illustrates a schematic diagram of the probe 104 including the shaft 105 and the one or more transducers 106. The one or more transducers 106 may include transducers designed to deliver FUS. The transducer(s) 106 may include one or more therapy transducers including therapy crystals and/or one or more image transducers including imaging crystals. As understood by those skilled in the art, the therapy transducers can include a fixed geometric focal spot or can include a focal spot that can be varied electronically, such as with an annular, linear, or phased array system. FIG. 1B illustrates a focal point and focal length 107 of transducer(s) 106.

The drive shaft 105 may be at least generally flexible, such as at one or more discrete segments thereof or along an entire length of the drive shaft 105. The one or more transducers 106 may be mounted at the end of drive shaft 105 in alignment with the drive shaft 105, or orthogonal to the end of the drive shaft 105, or in any other orientation or design that allows the transducer(s) 106 to be brought to bear on target tissue.

Optionally, a magnetic localization system fiber and sensor 107 and/or a means for optical imaging 108 comprising optical imaging and light source fibers can be mounted to the drive shaft 105 as well. The drive shaft 105 can be secured to an apparatus moving in the drive shaft 105 in linear and/or rotational fashion so as to adjust the position of the at least one transducer 106. By way of a non-limiting example, the shaft 105 can be connected to one or more motors that can be activated manually or under computer control to adjust the position of the probe 104 and the transducer(s) 106 in angular and/or linear directions. Additionally, computer or manual controlled movement can be used to insert the probe 104 through the sheath 101.

FIG. 1C illustrates a schematic diagram of the sheath 101. By way of a non-limiting example, sheath 101 may be constructed of a thin walled material, such as an acoustically transparent (ultrasound transparent) material, and may be large enough in internal diameter to allow for passage of the probe 104. The sheath 101 may be made of flexible or rigid materials or some combination of flexible and rigid materials. If the sheath 101 is constructed primarily of rigid materials, it should still have sufficient flexibility to allow it to bend or flex when inserted into a patient. If the sheath 101 is flexible, an internal obturator can be provided (as understood by those skilled in the art), which will stiffen the sheath 101 when the sheath 101 is introduced into a naturally occurring or surgically created opening in a patient and can then be removed after insertion. The obdurator can be removably coupled to the distal end (or proximate to the distal end) of the flexible sheath 101 and configured to block the passage of fluid out of the flexible sheath 101.

A distal end of the sheath 101 may include an opening 102 configured to allow the passage of at least some fluid between the sheath 101 and a chamber in a patient. An opposing proximal end of the sheath 101 is equipped with the one or more ports 103A, 103B for fluid passage into and out of the sheath 101. Although two ports 103A, 103B are shown in FIGS. 1A and 1C, it will be understood that the actual number of ports in the sheath 101 may be greater or fewer. By way of a non-limiting example, the one or more ports 103A, 103B may include a single port, or may include two or more ports for fluid passage, such as ingress and egress ports. The ports 103A, 103B may be integrated into the sheath 100 in a manner well known to those skilled in the art, such that fluid can be passed in and out of the end of the sheath 100.

Additionally, the ports 103A, 103B or fluid lines connected to the ports 103A, 103B can be equipped with motorized valves or pinch clamps or other similar means for opening and closing the ports or connecting fluid lines, thereby providing the option of automated control of the volume of fluid passing through the sheath 101 and into or out of the chamber in the patient. Additionally, the FUS device 100 can include a controller configured to adjust the volume of the chamber in the patient by controlling the passage of fluid between the sheath 101 and the chamber.

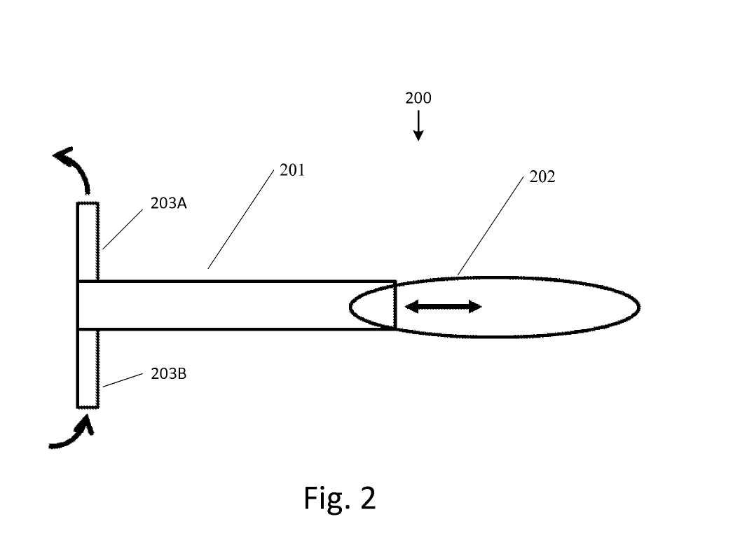

FIG. 2 illustrates another non-limiting embodiment of the sheath 201 of FUS device 200. The sheath 201 may include a balloon 202 coupled to the distal end of the sheath 201 and configured to exchange fluid with the sheath 201. As shown in FIG. 2, an interior of the balloon 202 may include an artificial chamber which can exchange fluid with the sheath 201. The balloon 202 may be constructed of an inflatable membrane or some other suitable material. By way of a non-limiting example, the coupling of the balloon 202 to the sheath 201 can be permanent, such that the opening in the distal end of the sheath 201 permanently opens into the interior of the balloon. Alternatively, the balloon can 202 be removably coupled to the sheath 201.

As shown in FIG. 2, the fluid ports 203A, 203B at the proximal end of the sheath 201 may connect to the inside of the balloon 202, allowing the balloon 202 to be filled with and distended by fluid. Thus, the size of the balloon 202 may be controlled by the relative amount of fluid that flows into and out of the balloon 202.

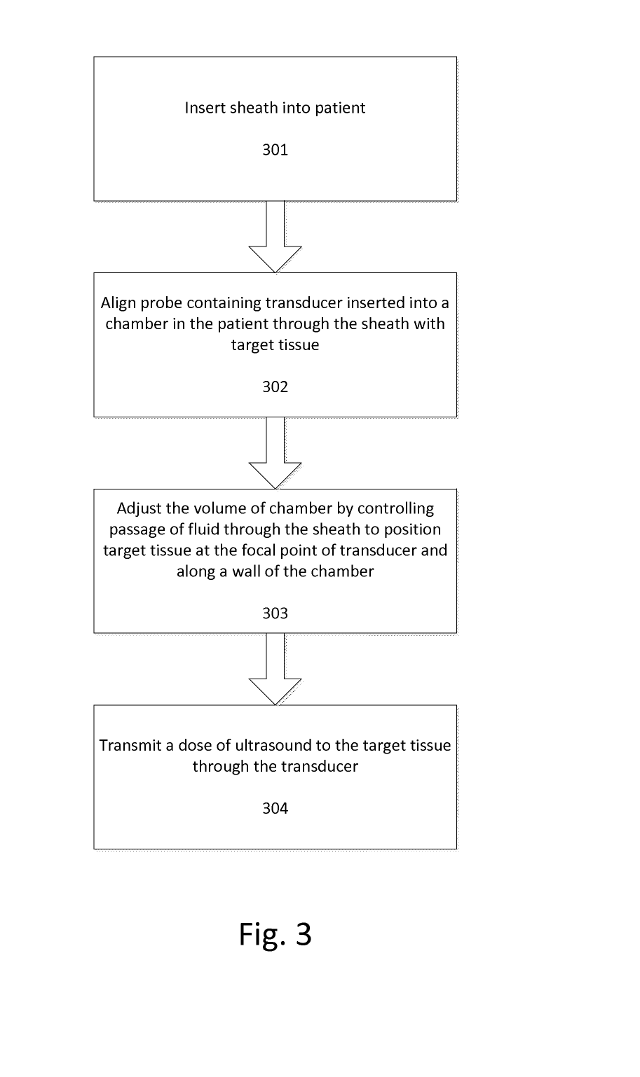

FIG. 3 illustrates a flowchart for delivering an ultrasound treatment according to an exemplary embodiment of the present disclosure. The method described by the flowchart can be implemented with any of the devices or variations of devices described in this application, and is not limited to the specific variations described with respect to the method of the flowchart.

At step 301 a sheath may be inserted into a patient. The sheath may include a distal end and a proximal end, the distal end including an opening configured to allow the passage of a fluid between the sheath and a chamber in the patient and the proximal end including one or more ports configured to allow the passage of a fluid into and out of the sheath.

At step 302 a probe may be inserted into the chamber through the sheath is aligned with a target tissue or target region. As discussed above, the probe may include a shaft and one or more transducers, such as ultrasound transducers, coupled to the shaft. Aligning can include translating the transducer(s) (and the shaft of the probe) in both angular and linear directions to align with the target tissue or target region.

At step 303 the volume of the chamber may be adjusted by controlling the passage of fluid between the sheath and the chamber to position the target tissue or target region at a focal point of the transducer(s). In the event that there are no natural chambers in the patient near the target tissue or target region, the sheath can include a balloon coupled to the distal end of the sheath as described earlier. In this case, the chamber is an artificial chamber comprising the interior of the balloon coupled to the distal end of the sheath.

Additionally, at step 304, a dose of ultrasound may be transmitted to the target tissue or target region through the one or more transducers that are part of the probe. One or more additional doses of ultrasound can also be transmitted after the initial dose. By way of a non-limiting example, the ultrasound transducer can be moved to a different position within the chamber, the volume of the chamber can be adjusted to a different volume by controlling the passage of fluid between the sheath and the chamber, and/or the one or more additional doses of ultrasound can be transmitted to the same or another target tissue or region through the one or more transducer(s).

By moving the transducer(s) to additional positions within the chamber by adjusting the position of the transducer, adjusting the volume of the chamber by adjusting the inflow/outflow of fluid from the chamber, and delivering additional doses of ultrasound therapy at the additional positions, a region of tissue larger than the focal spot of the transducer(s) can be treated without the need for a means for creating an acoustic window that is secured to or an integral part of the probe itself.

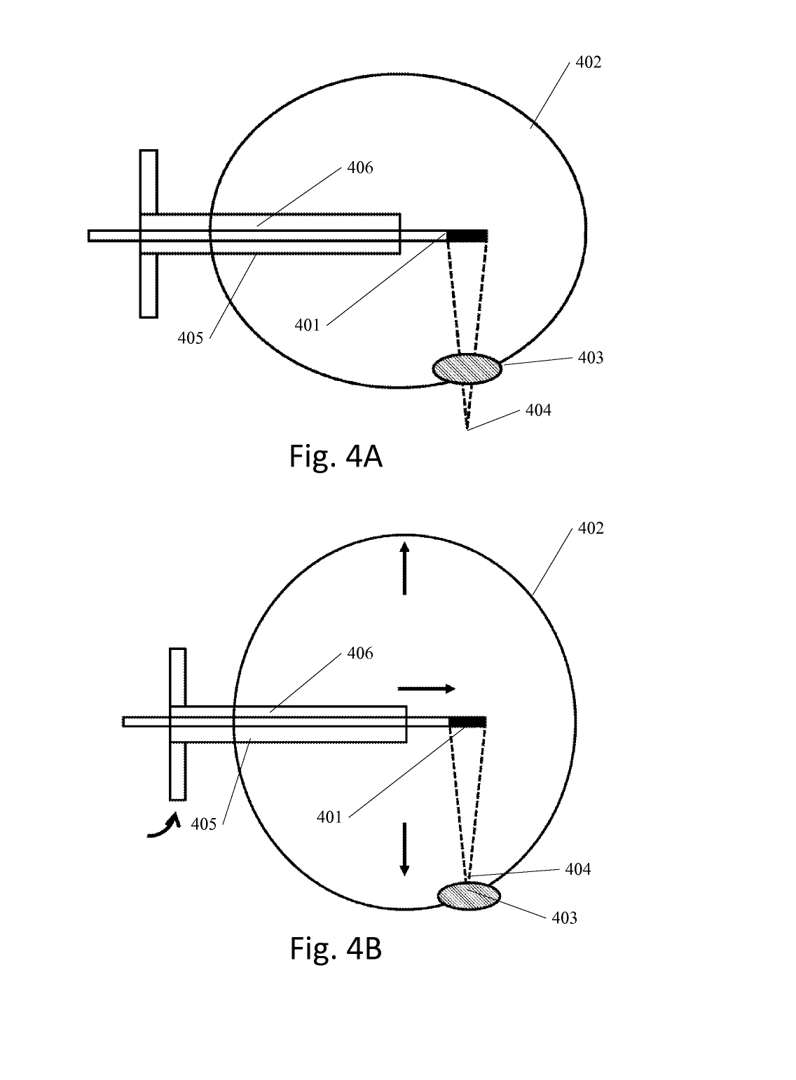

FIGS. 4A-4B are diagrams illustrating the operation of the focused ultrasound device in a natural chamber 402 according to an exemplary embodiment of the present disclosure. As shown in FIG. 4A, a sheath 405 has been inserted at least partially or completely into a chamber 402 in a patient. In FIG. 4A, the sheath 405 has been inserted into a naturally occurring chamber 402 in the patient that is filled with fluid. The sheath 405 may be a flexible sheath and may be inserted into the patient with an obturator, which is removed after insertion.

Also shown in FIG. 4A is a FUS probe 406 that has been inserted through the sheath 405 into the chamber 402. Visualization of the interior of the chamber 402 can be accomplished by an ancillary means of optic imaging such as fiber optics existing in the sheath 405, by optical imaging such as fiber optics incorporated into the probe 406 itself, or be an endoscope introduced into the chamber 402 through a separate portal.

US imaging of the interior of the chamber 402 can be achieved by an imaging crystal incorporated into the probe 406 or by an US imaging probe introduced into the chamber through a separate portal. It can also be achieved by radiological means of imaging including x-rays, MRI, and other 3-D volumetric means of imaging. The position of the probe 406 relative to the chamber 402 can be indicated by a magnetic or camera localization system which the probe 406 can be equipped with. Using any of the above means, upon identification of the region to be treated 403, the probe 405 is guided to the relatively correct treatment position.

In order to position the US therapy transducer(s) 401 such that the focal spot 404 of the transducer(s) when activated falls on the region to be treated 403, the distance between the transducer(s) 401 and the region to be treated 403 must be equal to the focal length of the transducer(s) 401. This distance may be determined by using the optical or US imaging system as a range finder to determine the depth of the chamber 402 wall relative to the transducer(s) 401.

As shown in FIG. 4B, the above-described distance can then be adjusted by controlling the flow of fluid into the chamber 402 through the ingress and egress lines that are part of the shaft 405. As shown in FIG. 4B, increasing the amount of fluid in the chamber 402 will cause the chamber 402 to enlarge, thereby increasing the distance of the region to be treated 403 from the transducer(s) 401. In contrast, decreasing the total amount of fluid in the chamber 402 will decrease the size of the chamber 402, thereby decreasing the distance of the region to be treated 403 from the transducer(s) 401. Once the distance of the region to be treated 403 from the transducer(s) 401 is equal to the focal distance 404 of the transducer(s) 401, the treatment can proceed. Thereby, the focal point 404 of the transducer(s) 401 is positioned correctly in the region to be treated 403 in the wall of the chamber 402 and acoustic interface with the target tissue in the region to be treated 403 is created. The distance can be kept constant by continuously or periodically during the course of treatment assessing the distance using the above means and correcting the distance as required.

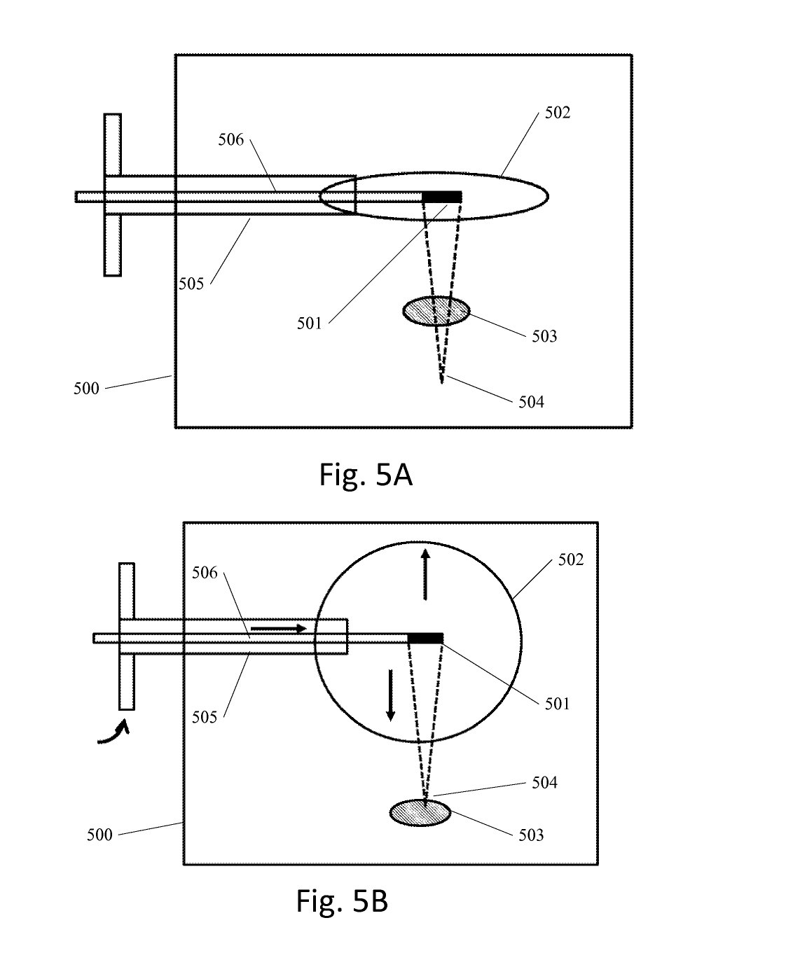

FIGS. 5A-5B are diagrams illustrating the operation of the focused ultrasound device in an artificial chamber according to an exemplary embodiment of the present disclosure. As shown in FIG. 5A, in cases where there is no naturally occurring chamber in the region to be treated 503, a chamber can be created by using a balloon (502). In this case, prior to inserting the probe 506 through the lumen of the balloon 502, fluid is introduced into the balloon 502 to enlarge it, thereby creating an artificial chamber (comprising the interior of balloon 502) within the tissue 500.

As shown in FIG. 5B, by adjusting the volume of the balloon 502 during the course of the treatment, the focal spot 504 of the transducer(s) 501 can be moved around in the tissue as required. As an added benefit of this approach the increase in local tissue pressure resulting from the inflation of the balloon 502 can help with hemostasis. An obturator may inserted into the sheath 505 having the balloon 502 attached at the distal end thereof. The sheath 505 may be inserted through a naturally occurring or surgically created portal into a patient 500 to a desired location as determined by imaging and/or a localization system coupled to the sheath 505 and/or obdurator. The obturator may be removed and the balloon 502 may be inflated with fluid. A FUS probe 506 may then be inserted through the sheath 505 into the artificial chamber 502 created by the interior of the balloon 502.

The probe 506 may be guided to the relatively correct treatment position inside the balloon 502 under visualization provided by one or more of an ancillary means of optic imaging such as an endoscope, optical imaging provided by the probe 506 itself, US imaging provided by the probe 506 itself, radiological means of imaging including x-rays, MRI, and other 3-D volumetric means of imaging, and/or a magnetic localization system coupled to the probe 506.

The treatment position relative to the chamber wall is achieved precisely by using the imaging system as a range finder to determine the depth of the chamber wall relative to the transducer(s) 501. As shown in FIG. 5B, the correct distance of the chamber wall from the transducer(s) 501, as required by the focal point 504 of the transducer(s) 501, is achieved by controlling the flow of fluid into the chamber through the ingress and egress lines that part of the sheath 505, increasing or decreasing the size of the balloon 502 and chamber as required. Thereby, the focal point 504 of the transducer(s) 501 is positioned correctly in the region to be treated 503 in the tissue and an acoustic interface with the target tissue in the region to be treated 503 is created.

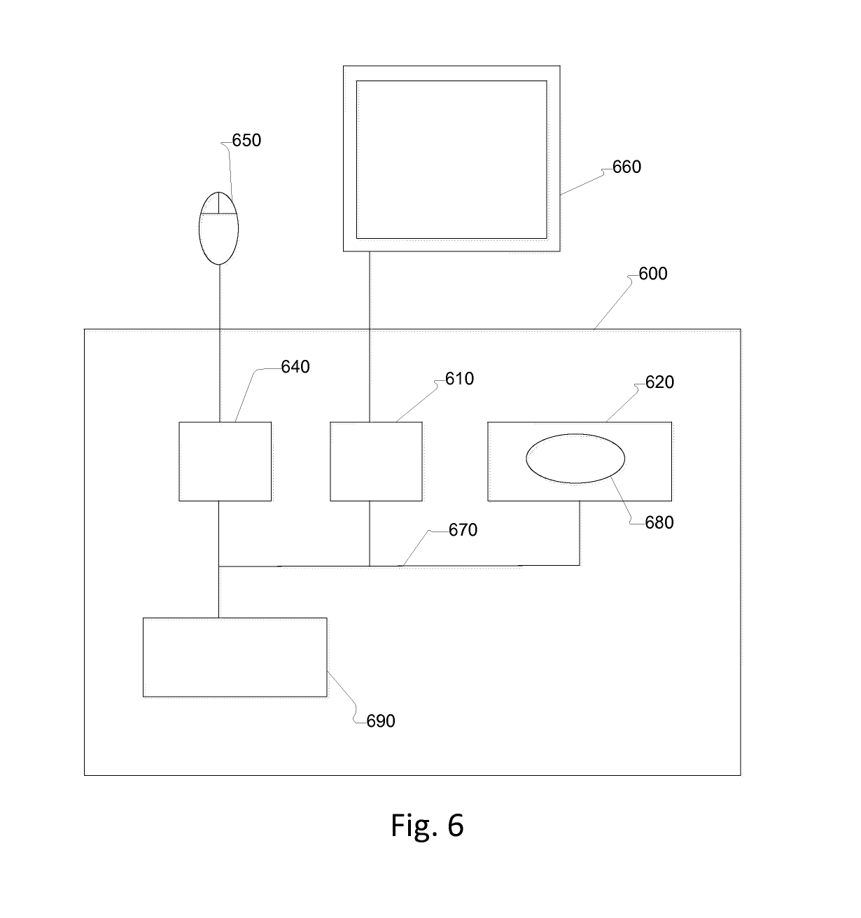

One or more of the above-described techniques may be implemented in or involve one or more computer systems. FIG. 6 illustrates a generalized example of a computing environment 600. The computing environment 600 is not intended to suggest any limitation as to scope of use or functionality of a described embodiment.

With reference to FIG. 6, the computing environment 600 includes at least one processing unit 610 and at least one memory 620. The processing unit 610 executes computer-executable instructions and may be a real or a virtual processor. In a multi-processing system, multiple processing units execute computer-executable instructions to increase processing power. The memory 620 may be volatile memory (e.g., registers, cache, RAM), non-volatile memory (e.g., ROM, EEPROM, flash memory, etc.), or some combination of the two. The memory 620 may store software instructions 680 for implementing the described techniques when executed by one or more processors. The memory 620 may be one memory device or multiple memory devices.

A computing environment may have additional features. For example, the computing environment 600 includes storage 640, one or more input devices 650, one or more output devices 660, and one or more communication connections 690. An interconnection mechanism 670, such as a bus, controller, or network interconnects the components of the computing environment 600. Typically, operating system software or firmware (not shown) provides an operating environment for other software executing in the computing environment 600, and coordinates activities of the components of the computing environment 600.

The storage 640 may be removable or non-removable, and includes magnetic disks, magnetic tapes or cassettes, CD-ROMs, CD-RWs, DVDs, or any other medium which may be used to store information and which may be accessed within the computing environment 600. The storage 640 may store instructions for the software 680.

The input device(s) 650 may be a touch input device such as a keyboard, mouse, pen, trackball, touch screen, or game controller, a voice input device, a smayning device, a digital camera, remote control, or another device that provides input to the computing environment 600. The output device(s) 660 may be a display, television, monitor, printer, speaker, or another device that provides output from the computing environment 600.

The communication connection(s) 690 enable communication over a communication medium to another computing entity. The communication medium conveys information such as computer-executable instructions, audio or video information, or other data in a modulated data signal. A modulated data signal is a signal that has one or more of its characteristics set or changed in such a manner as to encode information in the signal. By way of example, and not limitation, communication media include wired or wireless techniques implemented with an electrical, optical, RF, infrared, acoustic, or other carrier.

Implementations may be described in the general context of computer-readable media. Computer-readable media are any available media that may be accessed within a computing environment. By way of example, and not limitation, within the computing environment 600, computer-readable media include memory 620, storage 640, communication media, and combinations of any of the above.

Of course, FIG. 6 illustrates computing environment 600, display device 660, and input device 650 as separate devices for ease of identification only. Computing environment 600, display device 660, and input device 650 may be separate devices (e.g., a personal computer connected by wires to a monitor and mouse), may be integrated in a single device (e.g., a mobile device with a touch-display, such as a smartphone or a tablet), or any combination of devices (e.g., a computing device operatively coupled to a touch-screen display device, a plurality of computing devices attached to a single display device and input device, etc.). Computing environment 600 may be a set-top box, mobile device, personal computer, or one or more servers, for example a farm of networked servers, a clustered server environment, or a cloud network of computing devices.

Having described and illustrated the principles of our invention with reference to the described embodiment, it will be recognized that the described embodiment may be modified in arrangement and detail without departing from such principles. It should be understood that the programs, processes, or methods described herein are not related or limited to any particular type of computing environment, unless indicated otherwise. Various types of general purpose or specialized computing environments may be used with or perform operations in accordance with the teachings described herein. Elements of the described embodiment shown in software may be implemented in hardware and vice versa.

In view of the many possible embodiments to which the principles of our invention may be applied, we claim as our invention all such embodiments as may come within the scope and spirit of the following claims and equivalents thereto.

What is claimed is:

1. A system for delivering ultrasound, the system comprising:
a sheath having a distal end, an opposing proximal end and a passageway therebetween, the distal end including an opening configured to allow passage of fluid between the sheath and a chamber in a patient, the proximal end including one or more ports configured to allow the passage of the fluid into and out of the sheath; and
a probe including a shaft and at least one ultrasound transducer coupled to the shaft, the probe being configured for insertion into the proximal end of the sheath.

2. The system of claim 1, wherein the sheath is constructed from an ultrasound transparent material.

3. The system of claim 1, further comprising:
one or more motors coupled to the shaft, wherein the one or more motors are configured to adjust a position of the at least one ultrasound transducer in both angular and linear directions.

4. The system of claim 1, further comprising:
a balloon coupled to the distal end of the sheath, the balloon being configured to exchange fluid with the sheath.

5. The system of claim 1 further comprising:
a controller configured to adjust the volume of the chamber by controlling passage of fluid between the sheath and the chamber to position a target tissue at a focal point of the at least one ultrasound transducer.

6. The system of claim 1, further comprising an imaging device configured to capture an image of the chamber.

7. The system of claim 1, wherein the shaft comprises one of a flexible shaft and a rigid shaft.

8. The system of claim 1, wherein the one or more ports comprise at least one ingress port and at least one egress port.

9. The system of claim 1, further comprising one or more valves configured to open and close the one or more ports.

10. The system of claim 1, further comprising an obdurator removably coupled to the distal end of the sheath, the obdurator configured to block the passage of fluid out of the sheath.

11. A method of delivering ultrasound treatment, the method comprising:
inserting at least a portion of a sheath into a patient, the sheath comprising a distal end, an opposing proximal end and a passageway therebetween, the distal end including an opening configured to allow passage of fluid between the sheath and a chamber in the patient, the proximal end including one or more ports configured to allow passage of a fluid into and out of the sheath;
aligning at least a portion of a probe inserted into the chamber through the sheath with a target tissue, the probe comprises a shaft and at least one ultrasound transducer coupled to the shaft;
adjusting a volume of the chamber by controlling the passage of fluid between the sheath and the chamber to position the target tissue at a focal point of the ultrasound transducer; and
transmitting ultrasound to the target tissue through the at least one ultrasound transducer.

12. The method of claim 11, further comprising:
translating the ultrasound transducer in both angular and linear directions to align with the target tissue.

13. The method of claim 11, further comprising:
moving the ultrasound transducer to a different position within the chamber;
adjusting the volume of the chamber by controlling the passage of fluid between the sheath and the chamber; and
transmitting ultrasound to another target tissue through the at least one ultrasound transducer.

14. The method of claim 11, wherein the chamber is an artificial chamber comprising the interior of a balloon coupled to the distal end of the sheath.

15. The method of claim 11, wherein the shaft comprises one of a flexible shaft and a rigid shaft.

16. The method of claim 11, further comprising:
capturing an image of the chamber with an imaging device.

17. The method of claim 11, wherein the one or more ports comprise at least one ingress port and at least one egress port.

18. The method of claim 11, wherein the one or more ports are configured to open and close.

19. The method of claim 11, wherein the distal end of the flexible sheath is blocked by an obdurator removably coupled to the distal end of the flexible sheath.
